(19) United States
(12) Patent Application Publication
(10) Pub. No.: US 2024/0148739 A1
(43) Pub. Date: May 9, 2024

(54) METHOD OF DECREASING THE POPULATION OF FUSOBACTERIA IN THE GUT MICROBIOME

(71) Applicant: DSM IP Assets B.V., Heerlen (NL)

(72) Inventors: Thanh-Van PHAM, Kaiseraugst (CH); Ateequr REHMAN, Kaiseraugst (CH); Nicole SEIFERT, Kaiseraugst (CH); Robert STEINERT, Kaiseraugst (CH); Wilbert SYBESMA, Kaiseraugst (CH)

(21) Appl. No.: 18/550,642

(22) PCT Filed: Mar. 17, 2022
(86) PCT No.: PCT/EP2022/056947

§ 371 Date: Sep. 14, 2023

(30) Foreign Application Priority Data
Mar. 19, 2021 (EP) 21163744.2

(51) Int. Cl. A61K 31/525; A61K 31/015; A61K 31/355; A61K 31/375; A61P 31/04

(52) U.S. Cl. CPC A61K 31/525; A61K 31/015; A61K 31/355; A61K 31/375; A61P 31/04

(57) ABSTRACT

Delivery of antioxidants (Vitamin C, Vitamin E, riboflavin, and beta-carotene) directly to the large intestine were seen to decrease the population of Fusobacterium in the gut microbiome. An increased population of these bacteria is associated with Fusobacterium bacteremia as a co-morbidity with any one of: malignancy, dementia, chronic obstructive lung disease, diabetes, heart disease, alcoholism, disease(s) requiring dialysis, and stroke; colorectal cancer; digestive diseases including Ulcerative colitis; Crohn's disease; and/or Pediatric or adult inflammatory bowel disease; colorectal and other cancers, inflammatory bowel disease, adverse pregnancy-related conditions, and HIV.

### TECHNICAL FIELD

This invention relates to the use of a combination of riboflavin, Vitamin C, beta-carotene, and Vitamin E which, when delivered directly to the large intestine, can lower the population of Fusobacteria spp. in the gut microbiome. As Fusobacterium presence in the gut is related to adverse conditions such as autism, colorectal and other cancers, inflammatory bowel disease, adverse pregnancy-related conditions, and HIV, this invention also relates to methods of treatment, prevention, and/or amelioration of symptoms of the above as well as to Fusobacterium spp. bacteremia (FB) by direct delivery of the aforesaid combination, to both the general population and those at higher risk of developing FB.

### BACKGROUND OF THE INVENTION

Bacteremia is the presence of bacteria in the bloodstream. Various bacteria normally present in the mouth or gut can enter the bloodstream via different pathways, and in some cases cause serious infections in the brain, pericardium, heart, bones and joints.

Fusobacterium is a genus of an anaerobic elongated gram-negative rods which can inhabit the oral, gastrointestinal, upper respiratory tract and vaginal mucosae as part of the normal flora. Fusobacterium bacteremia (FB) Infections can occur through disruptions of mucosal surfaces from trauma, tumor, or prior infection, and then can progress to various diseases. There are multiple species identified, and the two most commonly associated with diseases are F. necrophorum and F. nucleatum. F. necrophorum causes periodontal disease, tonsillitis, peritonsillar abscess, and thrombophlebitis of the jugular vein (Lemierre syndrome), and is generally found in persons under the age of 40.

F. nucleatum is also an agent in gingival and periodontal diseases, but is additionally seen elsewhere in the body, and often related to serious diseases, including metastatic infections involving the brain, liver, joints, and heart valves. There is a link between it and the progression and severity of colorectal cancers. Unlike F. necrophorum, F. nucleatum bacteremia is generally associated with people over the age of 40. It has also been associated with intrauterine infections, premature birth, and inflammatory bowel disease are also related to invasive F. nucleatum infection. People having the following underlying conditions have a significant risk of FB: malignancy, dementia, chronic obstructive lung disease, diabetes, heart disease, alcoholism, disease requiring dialysis, and stroke.

It would be desirable to provide a means of preventing, treating, and ameliorating diseases associated with an increased population of Fusobacterium as well as FB in persons at risk, as well as in persons not generally prone to FB.

### SUMMARY OF THE INVENTION

It has been found, in accordance with this invention that antioxidants, such as the combination of Vitamin C, Vitamin B2, Vitamin E and beta-carotene, when delivered directly to the large intestine, can decrease the population of Fusobacterium spp. found in the gut, thus reducing the risk of an episode of Fusobacterium bacteremia (FB) in people at risk, and/or reducing the occurrence of, or severity of other adverse conditions caused or associated with the presence of Fusobacterium spp. In preferred embodiments, the Fusobacterium spp. is F. nucleatum and/or F. necrophorum; more preferably is it F. nucleatum.

Preferably, the antioxidants comprise at least one antioxidant selected from the group consisting of: Vitamin C, Vitamin E, riboflavin and beta-carotene, and mixtures thereof. Preferably at least two are chosen. More preferably at least three are chosen, and most preferably and for each indication discussed below, all the aforementioned are utilized.

Thus, one embodiment of this invention is a method of decreasing the population of a Fusobacterium spp. in a person's gut comprising directly delivering to the person's large intestine, at least one antioxidant selected from the group consisting of: Vitamin C, Vitamin E, riboflavin and beta-carotene, and mixtures thereof. Another embodiment of this invention is the use of an antioxidant which is formulated to be released in a person's large intestine and selected from the group consisting of: Vitamin C, Vitamin E, riboflavin and beta-carotene, and mixtures thereof, to decrease the population of a Fusobacterium spp. in a person's large intestine. Another embodiment is the use of an antioxidant comprising at least one antioxidant selected from the group consisting of: Vitamin C, Vitamin E, riboflavin and beta-carotene, and mixtures thereof, said antioxidant being formulated to be released in a person's large intestine, in the manufacture of a medicament which reduces the population of Fusobacterium spp. in a person's large intestine.

There are numerous conditions which have been associated with an increased Fusobacterium population, so another embodiment of this invention is lowering the Fusobacterium population in a person whose population is high. Thus, in another embodiment, the person receiving the directly delivered antioxidant is a person who is experiencing, or is at risk of experiencing a condition associated with an elevated population of Fusobacterium, where the condition is selected from the group consisting of:
a person over 40 years of age;
Fusobacterium bacteremia as a co-morbidity with any one of: malignancy, dementia, chronic obstructive lung disease, diabetes, heart disease, alcoholism, disease(s) requiring dialysis, and stroke;
a cancer selected from the group consisting of: colorectal cancer, pancreatic cancer, oral squamous cell cancer and gastrointestinal cancer;
a digestive disorder selected from the group consisting of: Ulcerative colitis, Crohn's disease, and pediatric or adult inflammatory bowel disease and acute appendicitis;
autism;
adverse pregnancy outcomes selected from the group consisting of preterm birth, intra-amniotic infection, stillbirth, neonatal sepsis, and hypertensive disorders of pregnancy) and
HIV (Human immunodeficiency virus) infection.

“Treatment” may include non-therapeutic treatments.

### BRIEF DESCRIPTION OF THE FIGURES

FIG. 1 shows the modified version of a continuous batch fermentation model (such as the SHIME, or TWINSHIME, or QuadSHIME provided by Prodigest, Technologiepark-Zwijnaarde 94, 9052 Gent, Belgium) which were used for the current study. St: Stomach vessel, SI: Small Intestine vessel, St/SI: vessel serving as stomach and small intestine, PC: Proximal colon and DC: Distal colon.

FIG. 2. Effect of antioxidant blend (AOB) and the prebiotic (FOS) treatments compared to a blank control (CTRL) on the abundance of Fusobacterium nucleatum in the distal colon for donor A (A) and donor B (B). * significant difference compared to the blank control (p<0.05).

### DETAILED DESCRIPTION OF THE INVENTION

### Definitions

As used throughout, the following definitions apply:

The term “riboflavin” which can be used interchangeably with “Vitamin B2”, includes riboflavin and esters thereof, in particular riboflavin-5′-phosphate.

The term “vitamin C” which can be used interchangeably with “ascorbic acid” also includes pharmaceutically acceptable salts thereof (e.g., sodium ascorbate and calcium ascorbate) and pharmaceutically acceptable esters thereof (in particular ascorbyl palmitate).

The term “β-carotene” refers to n-carotene or Provitamin A.

The term “vitamin E” includes four forms of tocopherols (alpha-Tocopherol, beta-Tocopherol, gamma-Tocopherol and delta-Tocopherol) and four forms of tocotrienols (alpha-tocotrienols, beta-tocotrienols, gamma-tocotrienols and delta-tocotrienols) The term “direct delivery” means that the antioxidant is administered in a form such that the antioxidant bypasses the stomach, but is present in the lower intestinal tract, including the large intestine, where it is available to the gut microbiome. Formulations which accomplish this include various delayed delivery and/or slow-release formulations.

Co-Morbidities with Fusobacterium Bacteremia Risks

F. nucleatum bacteremia is associated with specific co-morbidities, and persons suffering from one of the co-morbidities is at risk of also experiencing FB. Persons over the age of 40 are more prone to FB than younger people. Persons also at risk include those experiencing: malignancy, dementia, chronic obstructive lung disease, diabetes, heart disease, alcoholism, disease(s) requiring dialysis, and stroke. Thus, one embodiment of this invention is a method of reducing the risk of developing FB comprising directly delivering an antioxidant comprising at least one compound selected from the group consisting of: Vitamin C, Vitamin E, riboflavin and beta-carotene, and mixtures thereof to a person at risk of experiencing or who is experiencing a malignancy, dementia, chronic obstructive lung disease, diabetes, heart disease, alcoholism, disease(s) requiring dialysis, and stroke. Another embodiment is the use of an antioxidant comprising at least one compound selected from the group consisting of: Vitamin C, Vitamin E, riboflavin and beta-carotene, and mixtures thereof, formulated to be released in the large intestine, in a person experiencing or at risk of experiencing a malignancy, dementia, chronic obstructive lung disease, diabetes, heart disease, alcoholism, disease(s) requiring dialysis, and stroke, in order to prevent, lessen the risk of acquiring, and/or treat FB. Another embodiment is the use of an antioxidant comprising at least one compound selected from the group consisting of: Vitamin C, Vitamin E, riboflavin and beta-carotene, and mixtures thereof, formulated to be released in the large intestine of a person experiencing a malignancy, dementia, chronic obstructive lung disease, diabetes, heart disease, alcoholism, disease(s) requiring dialysis, and stroke, in the manufacture of a medicament to prevent, lower the risk of, or treating FB.

Cancers

In another embodiment, the person has or is at risk of developing colorectal cancer, pancreatic cancer, or oral squamous cell cancer. Fusobacterium invasion to host is associated to colorectal cancer carcinogenesis. Fusobacteria also interacts with host by expressing virulence factors. Fusobacterium attaches with many cell types such as epithelial cells, endothelial cells, fibroblast, natural killer cells etc. Thus, the antioxidant(s) intervention can be used to reduce the invasion of Fusobacteria, reducing the virulence and attachment to host cells.

Fusobacterium stimulates the inflammatory cytokines such as NF-kB, 116, 11-10 and 11-18, these inflammatory cytokines promote the growth of CRC. Thus, the antioxidant(s) intervention can be used to reduce the secretion of inflammatory cytokines.

For colorectal cancer, for example, these risks include: being overweight or obese, particularly in men; not being physically active; eating a diet high in red meats; having a low blood level of Vitamin D; being a long-term smoker; heavy alcohol use having a personal or family history of colorectal polyps or previous colorectal cancer; having a history of inflammatory bowel disease; having an inherited syndrome linked to colorectal cancer, such as Lynch syndrome or familial adenomatous polyposis; ethnicity (Afro-Americans and Ashkenazi Jews); and having type 2 diabetes. F. nucleatum is involved with the development of colorectal cancer, so decreasing the population of F. nucleatum in the gut can decrease the risk of the development of colorectal cancer, both in the general population and the people with an above-identified risk.

Thus, another embodiment is a method of reducing the risk of a person at risk developing colorectal cancer pancreatic cancer, or oral squamous cell cancer comprising directly delivering an antioxidant comprising at least one compound selected from the group consisting of: Vitamin C, Vitamin E, riboflavin and beta-carotene, and mixtures thereof to the large intestine of the person at risk. Another embodiment is a method of lengthening the time of onset of colorectal cancer, pancreatic cancer, or oral squamous cell cancer comprising directly delivering an antioxidant comprising at least one compound selected from the group consisting of: Vitamin C, Vitamin E, riboflavin and beta-carotene, and mixtures thereof to a person at risk of developing colorectal cancer. Another embodiment is the use of at least one antioxidant selected from the group consisting of: Vitamin C, Vitamin E, riboflavin and beta-carotene, and mixtures to treat or prevent the occurrence of colorectal cancer, pancreatic cancer, or oral squamous cell cancer in a person, wherein the antioxidant is delivered directly to the large intestine of the person. Another embodiment is the use of an antioxidant comprising at least one compound selected from the group consisting of: Vitamin C, Vitamin E, riboflavin and beta-carotene, and mixtures in the manufacture of a medicament which prevents or treats colorectal cancer, pancreatic cancer, or oral squamous cell cancer.

In another embodiment, the person has an early stage of colorectal cancer, pancreatic cancer, or oral squamous cell cancer, and the direct delivery reduces the Fusobacterium spp. which is associated with severity and progression of colorectal cancer, pancreatic cancer, or oral squamous cell cancer.

In another embodiment, the person has a gastrointestinal cancer, and the direct delivery of at least one antioxidant selected from the group consisting of: Vitamin C, Vitamin E, riboflavin and beta-carotene, and mixtures thereof reduces the Fusobacterium spp. which contributes to the chemo-resistance of gastrointestinal cancers. Thus, the direct delivery of an antioxidant of this invention can be used to treat or reduce the severity of gastrointestinal cancers.

Inflammatory Bowel Disease and Related Conditions

In another embodiment, the person has or is at risk of developing ulcerative colitis. Thus, another embodiment of this invention is a method of treating, preventing, or lessening the risk of a person developing ulcerative colitis comprising directly delivering an antioxidant comprising at least one compound selected from the group consisting of: Vitamin C, Vitamin E, riboflavin and beta-carotene, and mixtures thereof to the large intestine of the person. Another embodiment is the use of at least one antioxidant selected from the group consisting of: Vitamin C, Vitamin E, riboflavin and beta-carotene, and mixtures to treat or prevent the occurrence of ulcerative colitis in a person, wherein the antioxidant is delivered directly to the large intestine of the person. Another embodiment is the use of an antioxidant comprising at least one compound selected from the group consisting of: Vitamin C, Vitamin E, riboflavin and beta-carotene, and mixtures in the manufacture of a medicament which prevents or treats ulcerative colitis.

In addition to ulcerative colitis, an increased Fusobacterium population is associated with Crohn's disease as well as both pediatric and adult inflammatory bowel disease. Thus, another embodiment of this invention is a method of treating, preventing, delaying the onset, or lessening the severity of a symptom of a condition selected from the group consisting of: Crohn's Disease, pediatric inflammatory bowel disease, and adult inflammatory bowel disease comprising administering directly to the large intestine at least one antioxidant selected from the group consisting of: Vitamin C, Vitamin E, riboflavin, and beta-carotene to a person in need thereof. Another embodiment of this invention is the use of at least one antioxidant delivered directly to the large intestine for treating, preventing, delaying the onset, or lessening the severity of a symptom of a condition selected from the group consisting of: Crohn's Disease, pediatric inflammatory bowel disease, and adult inflammatory bowel disease. An additional embodiment is the use of at least one of Vitamin C, Vitamin E, riboflavin, and beta-carotene formulated to be delivered to the large intestine in the manufacture of a medicament for use in treating, preventing, delaying the onset, or lessening the severity of a symptom of a condition selected from the group consisting of: Crohn's Disease, pediatric inflammatory bowel disease, and adult inflammatory bowel disease.

The antioxidant(s) of this invention can be administered alone, but may also be administered in combination with other therapies if another adverse condition exists.

Fusobacterium nucleatum induces inflammation and suppressing host immunity. Decreasing the number of Fusobacterium by deliver vitamins to the large intestine will therefore improve immune function.

Autism

An increased Fusobacterium has also been associated with autism. Thus, another embodiment of this invention is a method of treating, preventing, delaying the onset, or lessening the severity of a symptom of autism comprising administering directly to the large intestine at least one antioxidant selected from the group consisting of: Vitamin C, Vitamin E, riboflavin, and beta-carotene to a person in need thereof. Another embodiment of this invention is the use of at least one antioxidant selected from the group consisting of: Vitamin C, Vitamin E, riboflavin, and beta-carotene delivered directly to the large intestine for treating, preventing, delaying the onset, or lessening the severity of a symptom of autism. An additional embodiment is the use of at least one of Vitamin C, Vitamin E, riboflavin, and beta-carotene formulated to be delivered to the large intestine in the manufacture of a medicament for use in treating, preventing, delaying the onset, or lessening the severity of a symptom of autism.

Adverse Conditions During Pregnancy

Various adverse conditions in pregnancy including preterm birth, intra-amniotic infection, stillbirth, neonatal sepsis, and hypertensive disorders of pregnancy have been associated with an increased population of Fusobacterium. Thus another embodiment of this invention is a method of treating, preventing, delaying the onset, or lessening the severity of an adverse pregnancy condition selected from the group consisting of: preterm birth, intra-amniotic infection, stillbirth, neonatal sepsis, and hypertensive disorders of pregnancy comprising: administering directly to the large intestine at least one antioxidant selected from the group consisting of: Vitamin C, Vitamin E, riboflavin and beta-carotene to a person in need thereof. Another embodiment of this invention is the use of at least one antioxidant selected from the group consisting of: Vitamin C, Vitamin E, riboflavin, and beta-carotene delivered directly to the large intestine for treating, preventing, delaying the onset, or lessening the severity of a symptom of an adverse pregnancy condition selected from the group consisting of: preterm birth, intra-amniotic infection, stillbirth, neonatal sepsis, and hypertensive disorders of pregnancy. An additional embodiment is the use of at least one of Vitamin C, Vitamin E, riboflavin, and beta-carotene formulated to be delivered to the large intestine in the manufacture of a medicament for use in treating, preventing, delaying the onset, or lessening the severity of a symptom of an adverse pregnancy condition selected from the group consisting of: preterm birth, intra-amniotic infection, stillbirth, neonatal sepsis, and hypertensive disorders of pregnancy.

Human Immunodeficiency Virus (HIV) Infection

One condition related to HIV is an elevated population of Fusobacterium. Thus, another aspect of this invention is a method of treating, preventing, delaying the onset, or lessening the severity of a symptom of HIV comprising administering directly to the large intestine at least one antioxidant selected from the group consisting of: Vitamin C, Vitamin E, riboflavin, and beta-carotene to a person in need thereof. Another embodiment of this invention is the use of at least one antioxidant selected from the group consisting of: Vitamin C, Vitamin E, riboflavin, and beta-carotene delivered directly to the large intestine for treating, preventing, delaying the onset, or lessening the severity of a symptom of HIV. An additional embodiment is the use of at least one of Vitamin C, Vitamin E, riboflavin, and beta-carotene formulated to be delivered to the large intestine in the manufacture of a medicament for use in treating, preventing, delaying the onset, or lessening the severity of a symptom of HIV.

Doses:

Preferably, Vitamin B2 can be administered in an amount such that its local concentration in the colon is at least 0.01 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. Specific dosages per day can range up to 200 mg/day, preferably 5-100 mg/day, more preferably from 10-50 mg/day.

Preferably, β-carotene is administered in an amount such that its local concentration in the colon is at least 0.1 g/L, preferably at least 0.15 g/L, most preferably at least 0.2 g/L. Preferred local concentrations in the colon range from about 0.05 g/L to about 0.4 g/L, more preferably from about 0.15 g/L to about 0.25 g/L One preferred dosage per day is up to 150 mg.

Preferably, vitamin E (50%) is administered in an amount such that its local concentration in the colon is at least 0.005 g/L preferably at least 0.05 g/L, most preferably at least 0.15 g/L. Preferred local concentrations in the colon range from about 0.005 g/L to about 2.5 g/L, more preferably from about 0.15 g/L to about 1.75 g/L. One preferred dosage per day is up to 1000 mg.

Preferably, Ascorbic Acid can be administered in an amount such that its local concentration in the colon is at least 0.05 g/L, preferably at least 0.1 g/L, most preferably at least 2 g/L. Preferred local concentrations in the colon range from about 0.05 g/L to about 1.5 g/L, more preferably from about 0.5 g/L to about 1 g/L, most preferably from about 0.8 g/L to about 0.9 g/L. Specific dosages per day can range up to 2000 mg/day, preferably 100-2000 mg/day; more preferably 200-1000 mg/day.

Preferably the antioxidants are present in a ratio of:

| Riboflavin | 0.5 to 2 |
|---|---|
| Ascorbic Acid | 4 to 15 |
| Vitamin E | 1 to 5 |
| Beta-Carotene | 0.5-3 |

More preferably, the ratio of Riboflavin/Ascorbic acid/Vitamin E/13-Carotene is 1.0/6.6/1.3/1.6. In preferred embodiments, the compositions are administered for an extended period time, such as for at least once per day for at least 3 days, at least a week, at least two weeks and at least 4 weeks.

The antioxidants are preferably administered in a formulation which allows the antioxidant to be released in the large intestine. Such forms are known in the art. Alternatively, and perhaps preferably for non-human administration, the animal is administered a high enough dose for the antioxidant to be present in the large intestine.

The following non-limiting Examples are presented to better illustrate the invention.

### EXAMPLES

### Example 1

The aim of this study was to compare the effect of directly delivered antioxidants to that of a prebiotic: Fructooligosaccharides (FOS). Two donors were selected for the long-term SHIME® experiment, where the impact of repeated intake of the test products was evaluated on the composition (as assessed via 16SrRNA gene sequencing) of the luminal gut microbiome.

Design of the SHIME® Experiment

The typical reactor setup of the SHIME® represents the gastrointestinal tract of the adult human. It has a succession of five reactors simulating the different parts of the human gastrointestinal tract. The first two reactors are of the fill-and-draw principle to simulate different steps in food uptake and digestion, with peristaltic pumps adding a defined amount of SHIME feed (140 mL 3×/day) and pancreatic and bile liquid (60 mL 3×/day), respectively to the stomach (V1) and small intestine (V2) compartment and emptying the respective reactors after specified intervals. The last three compartments simulate the large intestine. These reactors are continuously stirred; they have a constant volume and pH control. Retention time and pH of the different vessels are chosen to resemble in vivo conditions in the different parts of the colon. Upon inoculation with fecal microbiota, these reactors simulate the ascending (V3), transverse (V4) and descending (V5) colon. Inoculum preparation, retention time, pH, temperature settings and reactor feed composition have been described elsewhere. Upon stabilization of the microbial community in the different regions of the colon, a representative microbial community is established in the three colon compartments, which differs both in composition and functionality in the different colon regions.

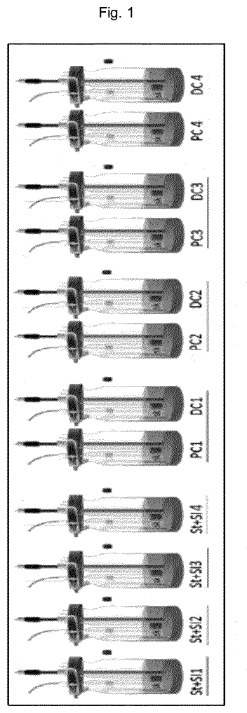

The conventional SHIME setup was adapted from a TWINSHIME configuration to a QuadSHIME® configuration (FIG. 1) allowing to compare four different conditions in parallel. During this specific project, the properties of three different test ingredients and a blank control were evaluated in a parallel TripleSHIME® configuration using the microbiota of two healthy adult human donors. As a compromise for the additional test conditions, the colon regions were limited to two regions as compared to three regions in the TWINSHIME. The retention times and pH ranges were optimized in order to obtain results that are representative of a full GIT simulation. In practice, in QuadSHIME® experiments, instead of working with 2 units, each composed of an AC-TC-DC configuration (ascending, transverse and descending colon), one used 4 PC-DC units. Upon inoculation with a faecal microbiota of a human adult, these reactors simulate the proximal colon (PC; pH 5.6-5.9; retention time=20 h; volume of 500 mL) and distal colon (DC; pH 6.6-6.9; retention time=32 h; volume of 800 mL).

The SHIME® experiment for this study consisted of two stages (Table 1, below):

Stabilization period: After the inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the fecal inoculum. Analysis of samples at the end of this period allows to determine the baseline microbial community composition and activity in the different reactors.

Treatment period: During this two-week period, the SHIME reactor was operated under nominal conditions, but with a diet supplemented with the test product. Samples taken from the colon reactors in this period allow to investigate the specific effect on the resident microbial community composition and activity. For the blank control condition, the standard SHIME nutrient matrix was further dosed to the model for a period of 14 days. Analysis of samples of these reactors allow to determine the nominal microbial community composition and activity in the different reactors, which will be used as a reference for evaluating the treatment effects.

TABLE 1
Overview of the different stages applied in this study.

| Week 1 | Week 2 | Week 3 | Week 4 |
|---|---|---|---|
| Stabilization | Stabilization | Treatment | Treatment |

Samples were collected at the following time points to follow up on the adaptation of the microbiota to the different test products:
Last three days of stabilization period;
Last two days of the first treatment week;
Last two days of the second treatment week.

Analysis of the Microbial Community Composition and Activity

An important characteristic of the SHIME is the possibility to work with a stabilized microbiota community and to regularly collect samples from the different intestinal regions for further analysis. The large volumes in the colonic regions allow to collect sufficient volumes of liquids each day, without disturbing the microbial community or endangering the rest of the experiment. A number of microbial parameters are monitored throughout the entire SHIME experiment. These measurements are necessary to evaluate the performance of the model and allow to monitor basic changes in the microbial community composition and activity due to the prebiotic treatment.

Microbial Community Composition

Samples were collected for 16S rRNA gene-targeted Illumina sequencing.

Analysis of the Microbial Community Composition

Two techniques were combined to map the community shifts induced by the different treatments in large detail:
16S rRNA gene-targeted Illumina sequencing, a PCR-based method by which microbial sequences are amplified until saturation, thus providing proportional abundances of different taxa at different phylogenetic levels (microbial phylum, family and OTU level). The methodology applied by ProDigest involves primers that span 2 hypervariable regions (V3-V4) of the 16S rDNA. Using a paired sequencing approach, sequencing of 2×250 bp results in 424 bp amplicons. Such fragments are taxonomically more useful as compared to smaller fragments that are taxonomically less informative.
Accurate quantification of total bacterial cells in the samples through flow cytometry. Combining the high-resolution phylogenetic information of the 16S rRNA gene-targeted Illumina together with the accurate enumeration of the cell count via flow cytometry, highly accurate, quantitative abundances of the different taxonomic entities inside the reactors can be obtained.

The comparisons of normally distributed data of the different stabilization and treatment weeks on microbial metabolic markers and microbial community parameters were performed with a Student's T-test assuming equal variance. Differences were considered significant if p<0.05.

Trial

Three different test products were tested in this project as compared to a blank control. The test products and the in vitro doses at which they were tested can be found in Table 2.

TABLE 2
List of test products and the in vitro dosage at which
they were tested in the long-term SHIME experiment.

| | | | In vitro |
|---|---|---|---|
| | | Product | dosage (mg/d) |
| Blend 1 | Non-prebiotic | Riboflavin | 75 |
| | vitamin | Ascorbic acid | 500 |
| | antioxidant | Dry Vitamin E (50% form) | 200 |
| | mix (AOB) | β-Carotene (10% form) | 1200 |
| Blend 2 | Prebiotic | XOS | 3000 |

Results:

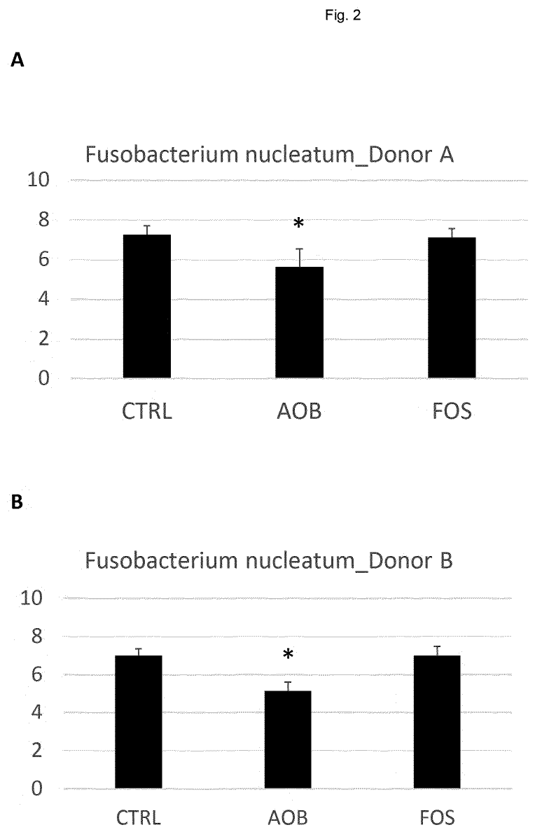

Decreased abundance of detrimental bacteria Fusobacterium nucleatum (FIG. 2)

Treatment of distal colon vessels with antioxidant resulted in significantly lower Fusobacterium nucleatum abundance compared to the control and prebiotic FOS in donor A and donor B.

1. A method of decreasing the population of a Fusobacterium spp. in a person's gut, comprising directly delivering to the person's large intestine, at least one antioxidant selected from the group consisting of: Vitamin C, Vitamin E, riboflavin, and beta-carotene, and mixtures thereof.

2. A method according to claim 1, wherein the person receiving the directly delivered antioxidant is a person who is experiencing, or is at risk of experiencing a condition associated with an elevated population of Fusobacterium selected from the group consisting of:
a person over 40 years of age;
Fusobacterium bacteremia as a co-morbidity with any one of: malignancy, dementia, chronic obstructive lung disease, diabetes, heart disease, alcoholism, disease(s) requiring dialysis, and stroke;
a cancer selected from the group consisting of: colorectal cancer, pancreatic cancer, oral squamous cell cancer and gastrointestinal cancer;
a digestive disorder selected from the group consisting of: Ulcerative colitis, Chron's disease, and pediatric or adult inflammatory bowel disease and acute appendicitis;
autism;
adverse pregnancy outcomes selected from the group consisting of preterm birth, intra-amniotic infection, stillbirth, neonatal sepsis, and hypertensive disorders of pregnancy; and
HIV (Human immunodeficiency virus) infection.

3. A method according to claim 1, wherein the antioxidants delivered directly to the large intestine comprise Vitamin C, Vitamin E, riboflavin, and beta-carotene.

4. Use of an antioxidant which is formulated to be released in a person's large intestine and selected from the group consisting of: Vitamin C, Vitamin E, riboflavin and beta-carotene, and mixtures thereof, to decrease the population of a Fusobacterium spp. in a person's large intestine.

5. Use according to claim 4, wherein the person is experiencing, or is at risk of experiencing a condition associated with an elevated population of Fusobacterium selected from the group consisting of:
a person over 40 years of age;
Fusobacterium bacteremia as a co-morbidity with any one of: malignancy, dementia, chronic obstructive lung disease, diabetes, heart disease, alcoholism, disease(s) requiring dialysis, and stroke;
a cancer selected from the group consisting of: colorectal cancer, pancreatic cancer, oral squamous cell cancer and gastrointestinal cancer;
a digestive disorder selected from the group consisting of: Ulcerative colitis, Chron's disease, and pediatric or adult inflammatory bowel disease and acute appendicitis;
autism;
adverse pregnancy outcomes selected from the group consisting of preterm birth, intra-amniotic infection, stillbirth, neonatal sepsis, and hypertensive disorders of pregnancy; and
HIV (Human immunodeficiency virus) infection.

6. Use according to claim 4, wherein the antioxidants delivered directly to the large intestine comprise Vitamin C, Vitamin E, riboflavin, and beta-carotene.
